SEQUENCE LISTING (3 sequences)

SEQ ID NO: 1 - DNA, 21 bp, Homo sapiens
aaggggaccacccgcactcgg

SEQ ID NO: 2 - DNA, 21 bp, Homo sapiens
aaggcaccaatttcgtcgtgg

SEQ ID NO: 3 - DNA, 21 bp, Homo sapiens
aagctcaacgagaatcactct

(19) United States
(12) Patent Application Publication
(10) Pub. No.: US 2008/0214799 A1
(43) Pub. Date: Sep. 4, 2008

(54) Biosynthetic platform for cardioprotective stress response in human fatal heart tissue

(76) Inventors: John G. Coles, Toronto (CA); Glen S. Van Arsdell, Toronto (CA); Mark Takahashi, Oakville (CA)

Correspondence Address: MCHALE & SLAVIN, P.A., 2855 PGA BLVD, PALM BEACH GARDENS, FL, 33410, US

(21) Appl. No.: 11/879,819

(22) Filed: Jul. 19, 2007

Related U.S. Application Data
(63) Continuation of application No. 10/833,543, filed on Apr. 27, 2004, now abandoned.
Application No. 10/833,543 is a continuation-in-part of application No. 10/429,656, filed on May 2, 2003.

(51) Int. Cl. C07H 21/00

(52) U.S. Cl. 536/23.5

(57) ABSTRACT

The present invention is directed toward elucidation of a human fetal gene expression program in response to simulated ischemia/reperfusion (I/R) in order to identify molecular targets which account for the innate cardioprotection exhibited by the fetal phenotype.

### REFERENCE TO RELATED APPLICATIONS

This application is a continuation of Ser. No. 10/833,543, filed Apr. 27, 2004, which is a continuation-in-part of Ser. No. 10/429,656, filed May 2, 2003, the contents of which are herein incorporated by reference.

### FIELD OF THE INVENTION

This invention relates to elucidation of the human fetal gene expression profile in response to simulated ischemia-reperfusion (I/R), in order to identify molecular targets which account for the innate cardioprotection exhibited by the fetal phenotype.

### BACKGROUND OF THE INVENTION

As indicated in our previous application, the global myocardial stress response during cardiac surgery has not been systematically studied or previously reported. Nor is it known whether there are age related differences in the stress response of the newborn heart in response to the stress of congenital heart surgery, or whether the response of neonatal myocardium is intrinsically different from that of older children.

This global gene expression profile of the heart in response to cardiac surgery has not previously been reported. The instant inventors hypothesized that the neonatal heart has an enhanced stress response due to a greater repertoire of inducible gene activation programs; and that the molecular basis of this response can be revealed by gene expression analysis of the immature (neonatal/fetal) heart which is pathologically stressed by congenital heart disease (CHD).

Neonatal myocardium has been found to exhibit a unique pattern of gene expression. This reflects a stress-induced protective program which includes both novel and precedented genes, which potentially represent important therapeutic targets.

The instant inventors have now determined that the human fetal cardiomyocyte exhibits resilience against pro-apoptotic stimuli. This was deemed evident by virtue of the relative attenuation of cardiomyocyte apoptosis, in comparison to that in a more mature cellular phenotype, in response to simulated ischemia-reperfusion and to exogenous IL-6 exposure. The transcript profile induced by simulated ischemia-reperfusion in the fetal CM reveals several putative molecular targets which may account for this innately cytoprotective phenotype. The instant inventors have previously shown that stress exposure elicits a compensatory stress-specific transcriptional response. This was based on the finding, in neonatal patients with hypertrophic congenital heart disease, of a dominant anti-hypertrophic transcriptional profile which appeared to be proportionate to the severity of hypertrophy. By analogy, therefore, it has now been shown that exposure of the human fetal CM to pro-apoptotic stimuli, specifically ischemia-reperfusion injury, can be used to identify compensatory anti-apoptotic molecular responses.

Furthermore, genome expression profiling in the fetal CM revealed a conspicuous repression, or lack of induction, of IL-6 transcription during ischemia and especially during reperfusion (58% of pre-ischemic levels by microarray; 25% by qPCR). IL-6 is a multifunctional cytokine with pro-proliferative and pro-hypertrophic properties in the heart. Up-regulation of serum and myocardial levels of pro-inflammatory cytokines [tumor necrosis factor (TNF)-α, interleukin-1; and IL-6] have been reported in infants with tetralogy of Fallot, and increased IL-6 message is found in ischemic/reperfused rat heart. IL-6 and its specific receptor (IL6Rα) are also up-regulated in the failing myocardium, and both are down-modulated in the process of favorable cardiac remodeling after left ventricular assist device implantation. However, the specific alterations in the IL-6 signaling pathway induced in the human CM during ischemia-reperfusion are unknown, and it is unresolved in the literature as to whether stress-induced elevation in circulating IL-6 represents a cardiomyocyte-protective or -injurious response.

### DESCRIPTION OF THE PRIOR ART

U.S. Pat. No. 6,365,352 relates to a method to identify granulocytic cell genes that are differentially expressed upon exposure to a pathogen or in a sterile inflammatory disease by preparing a gene expression profile of a granulocytic cell population exposed to a pathogen or isolated from a subject having a sterile inflammatory disease and comparing that profile to a profile prepared from quiescent granulocytic cells. The method is particularly useful for identifying cytokine genes, genes encoding cell surface receptors and genes encoding intermediary signaling molecules. The patent also includes methods to identify a therapeutic agent that modulates the expression of at least one gene in a granulocytic population. Genes which are differentially expressed during neutrophil contact with a pathogen, such as a virulent bacteria, or that are differentially expressed in a subject having a sterile inflammatory disease are of particular importance. The referenced patent relates solely to white blood cells and does not reference age-specific gene repertoires.

U.S. Pat. No. 6,461,814 provides a rapid, artifact free, improved method of obtaining short DNA “tag” or arrays thereof, allowing for determination of the relative abundance of a gene transcript within a given mRNA population and is useful to identify patterns of gene transcription, as well as identify new genes. This patent refers to a method of mRNA profiling which is not the subject of the present invention, nor does it infer the novel approach to elucidation of an age-related gene repertoire exploited by the instant invention.

Published Application US20010018182A1 is directed towards methods for monitoring disease states in a subject, as well as methods for monitoring the levels of effect of therapies upon a subject having one or more disease states. The methods involve: (i) measuring abundances of cellular constituents in a cell from a subject so that a diagnostic profile is obtained, (ii) measuring abundances of cellular constituents in a cell of one or more analogous subjects so that perturbation response profiles are obtained which correlate to a particular disease or therapy, and (iii) determining the interpolated perturbation response profile or profiles which best fit the diagnostic profile according to some objective measure. In other aspects, the invention also provides a computer system capable of performing the methods of the invention, data bases comprising perturbation response profiles for one or more diseases and/or therapies, and kits for determining levels of disease states and/or therapeutic effects according to the methods of the invention. The publication utilizes the concept of gene profiling to monitor disease states, however there is no conceptual overlap to the instant invention of obtaining a particular genetic response profile related to an age-related response to disease related stressors.

Published Application US20030008290A1 provides a method for serial analysis of gene expression, SAGE, a method for the rapid quantitative and qualitative analysis of transcripts, has been improved to provide more genetic information about each analyzed transcript. In SAGE, defined sequence tags corresponding to expressed genes are isolated and analyzed. Sequencing of over 1,000 defined tags in a short period of time (e.g., hours) reveals a gene expression pattern characteristic of the function of a cell or tissue. Although SAGE is useful as a gene discovery tool for the identification and isolation of novel sequence tags corresponding to novel transcripts and genes, the reference in no way contemplates the methodology practiced by the instant inventors.

Published Application US20030032030A1 teaches a method of measuring the biological age of a multicellular organism. In one embodiment, the method comprises the steps of: (a) obtaining a sample of nucleic acid isolated from the organism's organ, tissue or cell, wherein the nucleic acid is RNA or a cDNA copy of RNA and (b) determining the gene expression pattern of at least one of the genes selected from the group consisting of M21050, Z49204, U49430, K02782, X58861, X66295, M22531, X67809, U19118, M64086, M63695, U39066, X92590, X56518, AA182189, X16493, U20344, X16834, X82648, D00754, D16313, L38971 and X15789; and Published Application US20030036079A1 is drawn to a method of measuring the relative metabolic state of a multicellular organism is disclosed. In one embodiment, the method comprises the steps of: (a) obtaining a sample of nucleic acid isolated from the organism's organ, tissue or cell, wherein the nucleic acid is RNA or a cDNA copy of RNA, (b) determining the gene expression pattern of at least one of the genes selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088 and (c) determining whether the gene expression profile of step (b) is more similar to a CR-induced metabolic state or a standard diet metabolic state. Both US 23032030A1 and US 23036079A1 focus on senescence-related targets which can be used to retard aging, based on expression profiling in normal rodents; and do not involve stress response, as in the instant invention.

WO 09910535A1 teaches a method to identify stem cell genes that are differentially expressed in stem cells at various stages of differentiation when compared to undifferentiated stem cells by preparing a gene expression profile of a stem cell population and comparing the profile to a profile prepared from stem cells at different stages of differentiation, thereby identifying cDNA species, and therefore genes, which are expressed. Further disclosed are methods to identify a therapeutic agent that modulates the expression of at least one stem cell gene associated with the differentiation, proliferation and/or survival of stem cells. The disclosure's focus is on stem cell gene expression changes during hematopoiesis, not specifically the stress response; no overlap in targets exist with those of the instant disclosure.

WO09958720A1 provides methods for quantifying the relatedness of a first and second gene expression profile and for ordering the relatedness of a plurality of gene expression profiles to a single preselected gene expression profile. The methods are demonstrated to be useful for quantifying the relatedness of environmental conditions upon a cell, such as the relatedness in effects of pharmaceutical agents upon a cell. The methods are also useful in quantifying the relatedness of a preselected environmental condition to a defined genetic mutation of a cell and for quantifying the relatedness of a plurality of genetic mutations. Also presented are systems and apparatuses for performing the subject methods. Further provided are quantitative methods, systems, and apparatuses for selecting information subsets of genes for gene expression analysis. There is no contemplation of the utilization of neonatal or fetal cardiac tissue as a biosynthesis platform for cardioprotective gene expression.

U.S. Pat. No. 6,218,122 provides methods for monitoring disease states in a subject, as well as methods for monitoring the levels of effect of therapies upon a subject having one or more disease states. The methods involve: (i) measuring abundances of cellular constituents in a cell from a subject so that a diagnostic profile is obtained, (ii) measuring abundances of cellular constituents in a cell of one or more analogous subjects so that perturbation response profiles are obtained which correlate to a particular disease or therapy, and (iii) determining the interpolated perturbation response profile or profiles which best fit the diagnostic profile according to some objective measure. In other aspects, the invention also provides a computer system capable of performing the methods of the invention, data bases comprising perturbation response profiles for one or more diseases and/or therapies, and kits for determining levels of disease states and/or therapeutic effects according to the methods of the invention. The patent utilizes the concept of gene profiling to monitor disease states, however there is no conceptual overlap to the instant invention of obtaining a particular genetic response profile related to an age-related response to disease related stressors.

U.S. Pat. No. 6,406,853 is directed toward methods to screen interventions that mimic the effects of calorie restriction. Extensive analysis of genes for which expression is statistically different between control and calorie restricted animals has demonstrated that specific genes are preferentially expressed during calorie restriction. Screening for interventions which produce the same expression profile will provide interventions that increase life span. In a further aspect, it has been discovered that test animals on a calorie restricted diet for a relatively short time have a similar gene expression profile to test animals which have been on a long term calorie restricted diet. The effects of caloric restriction are not relevant to the instant invention.

U.S. Pat. No. 6,468,476 is directed toward bioinformatics methods for enhanced detection of biological response patterns. In one embodiment of the invention, genes are grouped into basis genesets according to the co-regulation of their expression. Expression of individual genes within a geneset is indicated with a single gene expression value for the geneset by a projection process. The expression values of genesets, rather than the expression of individual genes, are then used as the basis for comparison and detection of biological response with greatly enhanced sensitivity. In another embodiment of the invention, biological responses are grouped according to the similarity of their biological profile.

Published Application US20020064788A1 provides methods for identifying new compositions having one or more desired activities, and methods for identifying organisms that are sensitive or resistant to a drug composition. The methods are based upon genetic response profiles generated for an initial set of compositions, where at least one member of the set of compositions has been shown to have at least a first demonstrated activity and a second desired activity. By examining the patterns of genetic and cellular responses (i.e., the genetic response profiles) evoked by a first set of “known” compositions having varying degrees of one or both activities, a preferred pattern of genetic responses can be formulated which corresponds to the desired activity, but not to the demonstrated activity. Additional sets of compounds or compositions can then be screened for the desired genetic response profile, thereby identifying new compositions having the desired activity. Furthermore, populations of organisms can be screened for sensitivity or resistance to drug compositions, based upon comparison of genetic response profiles to the preferred pattern. The reference utilizes genetic profiles to look at responses to drug effects. This approach does not contemplate age-specific effects to identify beneficial targets, as is the case in the instant invention, nor does it contemplate the use of naturally-occurring, disease related stresses.

Published Application US20030036077 teaches methods for generating an mRNA expression profile from blood. In the subject methods, a population of nucleic acid targets is first generated from an acellular blood sample that contains a plurality of distinct mRNAs, i.e., a disease specific particular blood fraction. The resultant nucleic acid targets are hybridized to an array of nucleic acid probes to obtain an mRNA expression profile. The subject mRNA expression profiles are useful in the identification of disease specific markers. In such applications, the mRNA expression profiles are compared to a control expression profile to identify disease specific markers, where the identified markers subsequently find use in diagnostic applications. The subject methods also find use in diagnostic applications, where the mRNA expression profile is compared to a reference in making a diagnosis of the presence of a disease condition.

WO 00188188A2 provides for examining ischemic conditions, comprising measuring the expression levels of particular genes in a test sample or determining the expression profile of a gene group in the sample comprising a plurality of genes selected from said particular genes and is essentially a method to determine ischemia-inducible genes in tissues. This publication lacks the notion of disease-related stress, and the concept of exploiting the inherently greater protective response in young age exploited by the instant invention.

WO 09923254A1 Measures developmental changes in baseline (i.e; unstressed) gene expression, and thus is conceptually different from the instant invention.

### SUMMARY OF THE INVENTION

Human fetal cardiac myocytes exhibit a uniquely adaptive transcriptional response to ischemia-reperfusion which is associated with an apoptosis-resistance phenotype. The human fetal cardiomyocyte exhibits the capacity to inhibit stress-induced IL-6 signaling, as here demonstrated to depend upon regulation at both transcriptional and post-translational levels.

The idea that the immature heart has an inherently greater capacity to resist stress associated with hypoxia is supported by several investigations, although contradictory interpretations have been made which appear to be model-dependent. There is no PubMed-precedented information, however, regarding potential developmental changes in cardiomyocyte gene expression which might reveal the molecular mechanisms to account for the enhanced stress resistance in the immature human cardiac myocyte.

The idea that IL-6 pathway activation adversely affects cardiac function is solidly supported from clinical studies indicating that IL-6 and its specific receptor (IL-6Rα) are up-regulated in the failing myocardium, from the finding of increases in both IL-6, and the 130-kDa glycoprotein signaling subunit of the IL-6 receptor, gp130, at the mRNA and protein levels in the myocardium in patients with advanced heart failure in comparison to a control group, and by the large increases in IL-6 plasma concentration that occur during cardiopulmonary bypass. These studies, however, do not differentiate between the inference that cardiac stress engenders endogenous release of IL-6 as a protective response, and the diametrically opposed viewpoint that IL-6 is per se causative of cardiac damage.

Cellular responses to IL-6 are elicited by binding of soluble IL-6 to the transmembrane IL-6 receptor (IL-6Rα), which is followed by recruitment of two gp130 molecules into an active, multisubunit receptor complex. Homodimerization of gp130 triggers activation of several intracellular signaling pathways, which include the Janus kinase/Signal transducer and activator of transcription (JAK/STAT), Ras/mitogen-activated protein kinase (MAPK), and phosphatidylinositol 3-kinase (PI3-K) pathways. Thus, the IL-6 receptor complex consists of a ligand-binding molecule (IL-6Rα) and a signaling subunit, gp130, which provides a rapid membrane-to-nucleus signaling system regulating inflammatory gene expression.

We show here in preliminary expression profiling experiments that human fetal cardiac myocyte (HFCM) exposed to simulated ischemia with (I/R) or without reperfusion exhibit a uniquely adaptive transcriptional response. The “fetal” response includes a limited number of functional clusters dominated by predicted anti-inflammatory properties, featuring repression of IL-6 signaling, herein evident at both the mRNA and protein expression levels during reperfusion-mediated stress. These data provide a plausible and therapeutically important explanation for in the innately apoptotic-resistant human fetal CM phenotype.

Accordingly, it is a primary objective of the instant invention to use age-specific differential gene expression profiling for identifying protective genes which account for enhanced stress response in HFCM.

It is an additional objective of the instant invention to exploit the protective properties of the fetus using gene profiling of the fetal stress response as a result of exposure to simulated ischemia with or without reperfusion (I/R) in vitro.

It is still a further objective of the instant invention to exploit the enhanced fetal stress response to exogenous (artificial) stimuli designed to provoke a beneficial genetic response, including but not limited, to UV irradiation, environmental toxins, pathogenic organisms, and other noxious stimuli.

It is yet an additional objective of the instant invention to identify a useful biosynthetic platform for the identification of innate cardioprotective responses at the molecular level. The adaptive stress response exemplified by the fetal cardiomyocyte implicates the IL-6 pathway as an important and therapeutically-relevant arbiter of survival.

It is still an additional objective of the instant invention to define an expression strata of significant genes evidenced as a result of (I/R) in fetal heart tissue.

These and other objectives and advantages of the instant invention will become apparent from the following description wherein are set forth, by way of illustration and example, certain embodiments of this invention.

### BRIEF DESCRIPTION OF THE FIGURES

The patent or application contains at least one drawing executed in color. Copies of this patent or patent application publication with color drawings will be provided by the Office upon request and payment of the necessary fee.

FIG. 1: Heat map with hierarchical clustering of genes showing coherent expression patterns during ischemia (Isch) and reperfusion (R). Columns represent each of 2 different biological replicates each performed with 2 (dye-swapped) array replicates at each of the indicated time points. Red colour indicates higher expression, and green colour indicates lower expression. Gene-wise clustering reveals 4 temporally distinct expression strata: A: Repression during Isch and R; B: Repression during Isch; C: Activation during Isch and/or R; and D: Activation during Isch; repression during R. The far right column contains the Unigene cluster ID, the annotation for which is available at: http://genome-www5.stanford.edu/cgi-bin/source/sourceSearch. Genes with significantly different expression values from control are indicated in FIG. 5;

FIG. 2: Primary cultures of human fetal (HFCM) and 2-3 day neonatal rat (NRCM) were exposed to simulated ischemia with or without ‘reperfusion’ for the indicated time intervals. Cardiomyocytes were stained with 1 ug/mL Hoescht 33342 for detection of apoptotic nuclei based on typical pyknotic nuclear morphology, and the results expressed as fold change in the ratio of apoptotic to normal nuclei relative to that in control levels. The rates of apoptosis increased significantly with increasing duration of ischemia-reperfusion and were higher in the NRCM compared to HFCM [p(ANOVA)<0.05]. I, ischemia; R, reperfusion;

FIG. 3A: Western blot analysis was performed using lysates from human fetal cardiomyocytes at control (Ctrl), following 6 hr. simulated ischemia (Isch), and 3 hr. reperfusion (Isch/Rep), with and without addition of recombinant IL-6 (250 ng/ml) at the onset of Isch, as indicated at the top of each lane. Immunoblots were performed using total or phospho-specific antibodies against components of the IL-6 signaling cascade as indicated, and taken together, the results reveal deactivation of IL-6 signaling during ischemia-reperfusion as discussed in the text. Total STAT-3 and GSK-3β expression bands indicate equal protein loading in each lane which was also confirmed using actin controls. The results shown here represent three experiments exhibiting similar effects;

FIG. 3B: Western blot analysis was performed using lysates from human fetal cardiomyocytes at control (Ctrl), following 10 and 24 hr. of simulated ischemia (Isch), and 10 hr. reperfusion (Isch/Rep). Immunoblots were performed using both total and phospho-specific antibodies against PKB/Akt, MAPK and SAPK. The results indicate that deactivating dephosphorylation of PKB/Akt and MAPK occurs during ischemia with rephosphorylation evident following reperfusion, whereas the opposite phosphorylation events occur with SAPK. The results shown here represent three experiments exhibiting similar effects. Abbreviations are given in the text;

FIG. 4: Neonatal rat cardiomyocytes were transduced with lipofectamine only or lipofectamine containing ILK-targeted siRNA, exposed to 6 hr. ischemia and 3 hr. of reperfusion (I/R), and the relative rates of apoptosis quantitated using Hoescht 33342 staining as described in the text. Exposure of ILK-silenced NRCM to 6 hr. ischemia and 3 hr. reperfusion resulted in an approximate 50% decrease (p=0.031) in the apoptosis rate in comparison to lipofectamine-only controls (Ctrl). As shown in the insert, lipofectamine-mediated transfection of the ILK-specific siRNAs resulted in substantial (42%; p=0.02) knockdown of ILK expression as determined by Western blot analysis at 72 hr. post-transfection;

FIG. 5: Lists of differentially expressed genes during ischemia and reperfusion identified using SAM as described in the text. Unsupervised hierarchical clustering reveals four distinct expression strata as shown in FIG. 1. Fold changes are based on measurements at 4 hr. ischemia and 2 hr. of reperfusion compared to control levels. The far left column contains the Unigene cluster IDs, the annotations for which are available at: http://genome-www5.stanford.edu/cgi-bin/source/sourceSearch.

### DETAILED DESCRIPTION OF THE INVENTION

It is proposed that the fetal heart is highly resilient to hypoxic stress. Thus, the instant objective is to elucidate the human fetal gene expression profile in response to simulated ischemia- reperfusion (I/R), in order to identify molecular targets which account for the innate cardioprotection exhibited by the fetal phenotype.

Methods: Primary cultures of human fetal cardiac myocytes (HFCM) (gestational age 15-20 weeks) were exposed to simulated I/R in vitro using ischemic buffer and anoxic conditions. Total RNA from treated and baseline cells were isolated, reverse transcribed, and labeled with Cy3 or Cy5, and hybridized to a human cDNA microarray for expression analysis. This analysis revealed a highly significant (false discovery rate <3%) repression of interleukin-6 (IL-6) transcript levels during the reperfusion phase, confirmed by quantitative PCR (0.25+/−0.11-fold). IL-6 signaling during I/R was assessed at the protein expression level by Western measurements of IL-6 receptor (IL-6R), the signaling subunit of the IL-6R complex, gp130, and signal transducer of activated transcription-3 (STAT-3). Post-translational changes in the protein kinase B (PKB/Akt) signaling pathway were determined based on the phosphorylation status of PKB/Akt, mitogen-activated protein kinase (MAPK), and glycogen synthase kinase-3β (GSK-3β). Endogenous secretion of IL-6 protein in culture supernatants was measured by ELISA. The effect of suppression of a pro-hypertrophic kinase, integrin-linked kinase (ILK), using small-interfering (si) RNA was determined in an I/R-stressed neonatal rat cardiac myocyte (NRCM) model.

Results: HFCM exhibited a significantly lower rate of apoptosis induction during ischemia-reperfusion, and following exposure to staurosporine and recombinant IL-6, compared to that in neonatal rat CM [p(ANOVA)<0.05 for all comparisons]. The fetal transcriptional profile revealed 4 temporally distinct expression strata featuring suppression of IL-6 and mitogen-activated protein kinase 1 (MAPK), suggesting I/R-induced acquisition of an anti-inflammatory and anti-proliferative phenotype, and confirmed by coincident suppression of gp130 expression and STAT-3 phosphorylation during I/R. Exposure to exogenously-added recombinant IL-6 increased the apoptotic rate in both rat and human fetal CM (p<0.05). siRNA-mediated suppression of ILK, a pro-hypertrophy upstream kinase regulating PKB/Akt and GSK-3β phosphorylation, was cytoprotective against I/R-induced apoptosis in NRCM (p<0.05).

Conclusions: Human fetal CM exhibit a uniquely adaptive transcriptional response to ischemia-reperfusion which is associated with an apoptosis-resistance phenotype. The ‘fetal’ response features repression of IL-6 signaling and acquisition of a quiescent phenotype, which may serve the energetically beneficial purpose of dampening agonist-induced, pro-inflammatory and pro-proliferative signaling during I/R. The stress-inducible fetal CM gene repertoire is a useful platform for identification of targets relevant to the mitigation of cardiac ischemic injury, and highlights a novel avenue involving IL-6 modulation, for preventing cardiac myocyte injury associated with ischemia and reperfusion.

In accordance with this invention, immature heart tissue is understood to mean myocardial samples taken from patients within the age groupings as set forth above, as well as fetal myocardial tissue.

In accordance with this invention, the terms expression strata of significant genes, cardioprotective gene network, cardioprotective gene pattern, cardioprotective gene profile, and cardioprotective gene program are understood to mean a combination of nucleic acid sequences which are up-regulated and down-regulated in neonatal or fetal heart tissue as a result of naturally occurring disease states, e.g. naturally occurring and chronic hemodynamic and/or hypoxic stress, such as that induced by obstructive congenital heart disease.

In accordance with the present invention “Characteristic differentially expressed cardiac nucleic acid sequencing profile” refers to the difference in nucleic acid expression based on analysis of the patient myocardial sample, with direct comparison to normal values determined for a specific laboratory, or in comparison to corresponding data obtained from the same patient at an earlier time point in the clinical course of his disease. Such comparisons are facilitated by the method used in the current invention in which the transcript intensity corresponding to each probe on the array was compared to that corresponding probe in Universal Human RNA Reference sample. Other comparisons which may be informative would include those obtained through in silico database searches consisting of cardiac disease-specific transcriptional profiles.

### METHODOLOGY

### Cardiac Myocyte Cultures

Primary cultures of human fetal cardiac myocytes (HFCM) (gestational age 15-20 weeks) obtained under an Institutional Review Board-approved protocol were exposed to simulated ischemia with or without reperfusion (I/R) in vitro for the indicated time intervals using ischemic buffer and anoxic conditions, and a similar protocol was used for the isolation and culture of day 2-3 neonatal rat CM (NRCM).

### Microarray Gene Expression Analysis

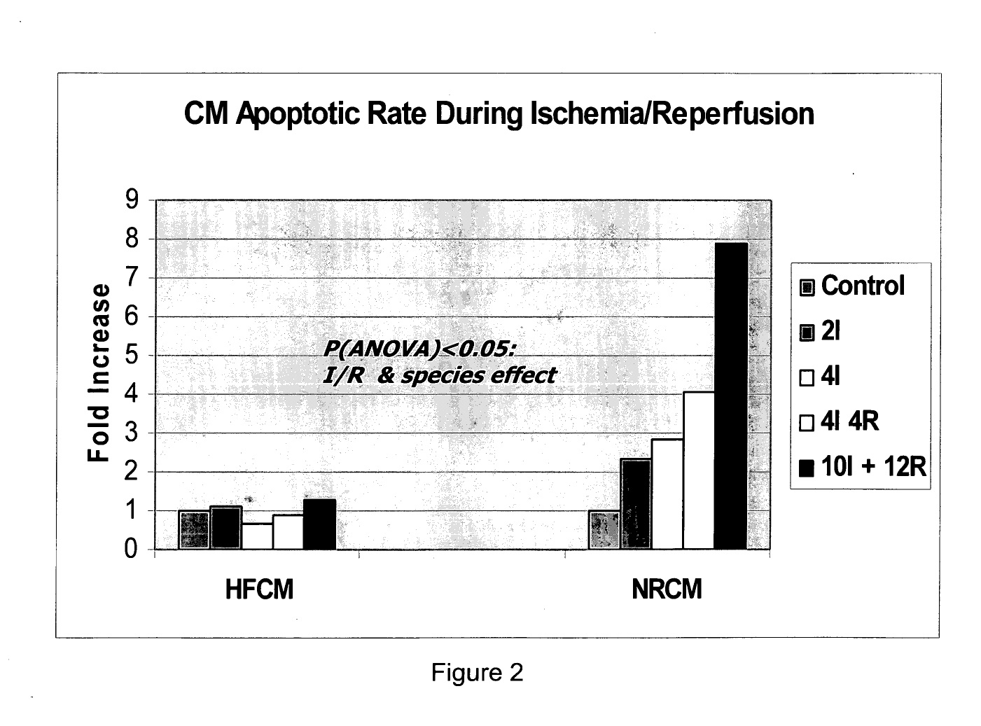

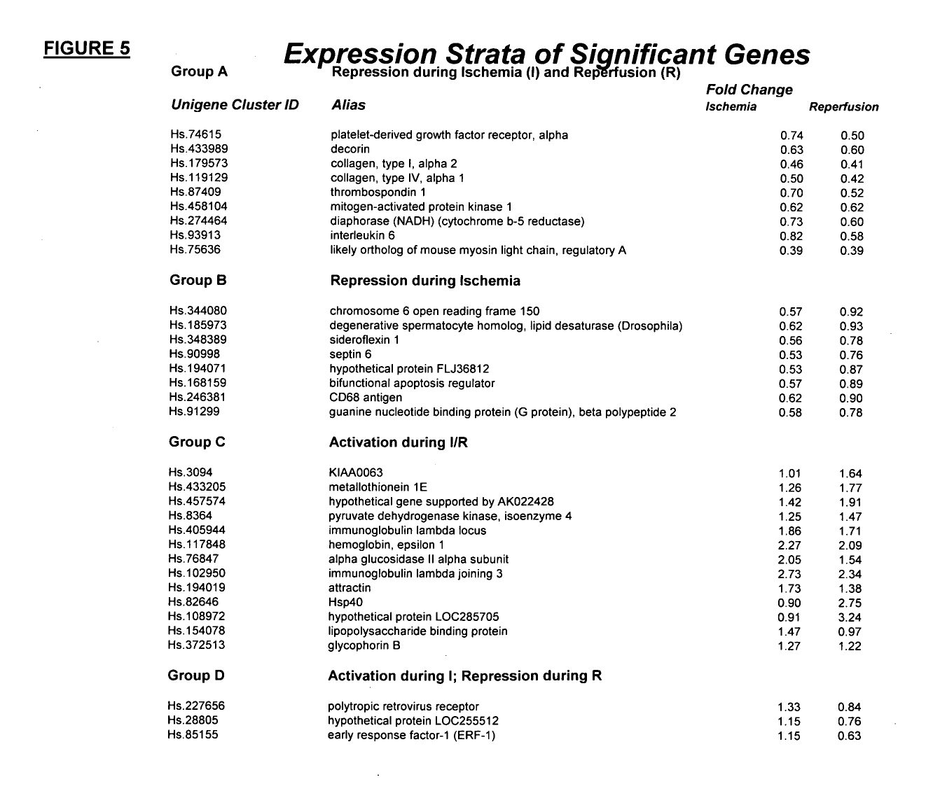

RNA isolation, fluorescence-labeling of cDNA, hybridization to spotted arrays containing 15,264 sequence-verified cDNA clones, and quantitative fluorescence scanning of gene expression intensity, were performed at the University of Toronto Health Network Microarray Centre (www.microarray.ca), as previously reported by us and others (for a list of publications see: http://www.microarrays.ca/about/pub.html). Significance of changes in sequential gene expression in HFCM exposed to I/R (at control; 4 hr. ischemia; and 4 hr. ischemia plus 2 hr. reperfusion) were determined by repeated permutation of MIAME-compliant (www.mged.org) data using Significance Analysis for Microarray (SAM). Results from the SAM analysis were visualized as hierarchical clusters in Gene Traffic (www.iobion.com) and significant genes classified by their differential response to ischemia and/or reperfusion. The results shown in FIG. 5 and FIG. 2 are based on 2 biological and 2 technical (array) replicates at each indicated time point with a false discovery rate (FDR), indicative of the statistical risk of incorrect identification of differentially-expressed genes, set to <3%.

### Validation Using qPCR

Independent confirmation of changes in IL-6 transcription levels was performed using real-time quantitative polymerase chain reaction (qPCR) as previously described by us. Primers were constructed against the 3′ ends of IL-6 and amplicon abundance determined in real-time by SYBR Green Dye (Applied Biosystems) fluorescence measurement during the logarithmic phase and normalized to that of a control gene, cyclophilin. Fold changes of the cyclophilin-normalized value of IL-6 transcript were determined as a ratio of cardiac myocyte culture-derived sample to that of the Universal Human Reference RNA.

### Western Blot Analysis

Fetal cardiomyocyte extracts containing 20 μg of protein were subjected to SDS/PAGE with 10% polyacrylamide gel and transferred onto Immobilon-P transfer membranes (Millipore). Analysis was performed with polyclonal PKB antibody (Transduction Laboratories), polyclonal Serine437 (S437) catalytically active, phosphorylation-specific PKB antibody (Cell Signaling Technology), polyclonal ILK antibody (Upstate Biotechnology), and anti-IL-6 receptor (IL-6Rα) and anti-gp130 antibodies (Santa Cruz Biotech). Monoclonal antibodies used for the determination of total and phosphorylated GSK-3β protein levels were from Biosource; total and phosphorylated (Py705) STAT-3, (Thr202/Tyr204) MAPK^{42/44}, and stress-activated protein kinase (SAPK-Thr183/Pyr185), were from Cell Signaling.

### IL-6 Measurements

IL-6 concentrations in the culture supernatants were determined using an enzyme-linked immunosorbent assay (ELISA) kit according to the manufacturer's instructions (Diaclone). The absorbance at 450 nm was measured and concentrations were determined by interpolation of a standard calibration curve. The lower limit of detection of IL-6 was 0.78 pg/mL. Human recombinant IL-6 was from Sigma (1-1395).

### Measurement of Apoptosis

Apoptosis of variously treated cardiomyocytes was determined based on nuclear condensation using Hoechst staining. Cardiomyocytes were stained with 1 ug/mL Hoescht 33342 trihydrochloride trihydrate (Molecular Probes) for detection of aopototic nuclei. Dishes were analyzed at 20× magnification using a Leica inverted deconvolution microscope with a coupled camera. Apoptotic cells were identified by their increased fluorescence due to chromatin condensation and pyknotic morphology. A minimum of 300 nuclei were counted per field and each data point consisted of four randomly selected fields. The measurement of CM apoptosis using Hoechst staining was found to correlate with, but was more sensitive than, that based on TdT-mediated dUTP nick-end labeling (TUNEL) labeling with the APO-BRDU kit and enumeration by flow cytometry (FACScan/CELL Quest system; BD Biosciences), and that based on Western measurement of cleaved caspase-3 Asp175 antibody (Cell Signaling). Percent apoptosis was determined as the ratio of apoptotic nuclei/total Hoechst-positive nuclei, and statistical comparisons made using Openlab 3.1.5 software. Statistical evaluation of intervention and cell type effects relied on a paired t-test or one-way ANOVA. Data are expressed as +/−SEM.

### Synthesis And Transfection of ILK-Specific Short Interfering RNA Molecules (siRNA)

Single-stranded siRNA were transcribed and annealed using a commercial kit, as outlined in the supplier's manual (Silencer Kit, Ambion). The following sequences were used to construct ILK siRNA: ILK1: 50-AAGGGGACCACCCGCACTCGG-30 SEQ ID NO: 1; ILK2: 50-AAGGCACCAATTTCGTCGTGG-30 SEQ ID NO: 2; and ILK3: 50-AAGCTCAACGAGAATCACTCT-30 SEQ ID NO: 3.

Each sequence was confirmed as unique using the BLAST algorithm. The specificity of ILK siRNA targeting vector has been previously shown. GAPDH control siRNA was provided with the Silencer siRNA construction kit. Transient transfections of neonatal rat cardiomyocytes were carried out using 6 μl of Lipofectin reagent (Invitrogen), according to the manufacturer's instructions. To quantitate the extent of knockdown of ILK protein, horseradish peroxidase-conjugated IgG was used as a secondary antibody, and ILK immunocomplexes visualized with an enhanced chemiluminescence (ECL) detection reagent (Amersham Pharmacia Biotech) and quantified by densitometry.

### Results

### Gene Expression Analysis Reveals An Anti-Inflammatory Transcriptional Response In HFCM

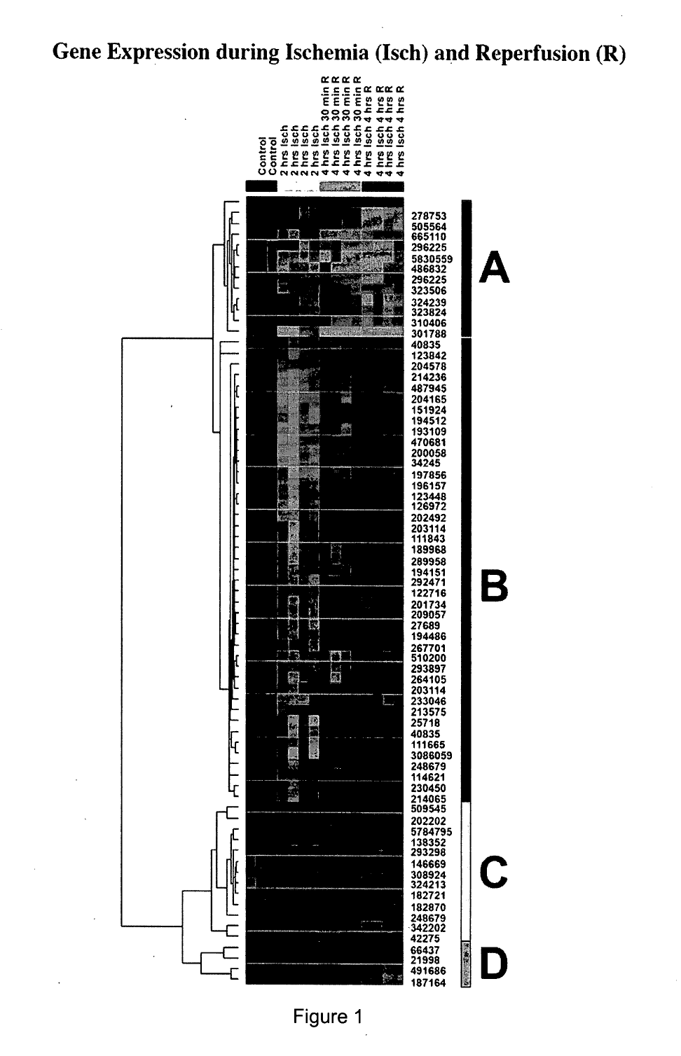

Gene-wise clustering shown in FIG. 1 reveals four temporally distinct expression strata:

A: Repression during ischemia and reperfusion; B: Repression during ischemia; C: Activation during ischemia and/or reperfusion; and D: Activation during ischemia and repression during reperfusion. The annotation of significant genes and corresponding expression values are indicated in FIG. 5. Noteworthy was the significant repression of IL-6 transcription during ischemia and especially during reperfusion: 58% of pre-ischemic levels by microarray analysis, and 25% +/−11% by qPCR measurement.

### The Fetal Cardiomyocyte Is Resistant To Apoptotic Stimuli

The rate of apoptosis measured using Hoechst staining shown in FIG. 2 was significantly lower in the fetal CM (relative to that in neonatal rat-derived CM) in response to increasing duration of ischemia with or without of reperfusion [p(ANOVA)<0.05 for rat vs human CM]. Exogenous IL-6 (250 ng/ml) caused a similar, approximately 3-4 fold increase in apoptosis, maximal at 3 hr. exposure, in both neonatal rat (p=0.012) and human fetal CM (p=0.034) during normoxia, and resulted in a significant increase in the apoptotic rates in both cellular phenotypes following 6 hr. ischemia (p<0.05); IL-6-mediated fold increases in apoptotic rates were greater in neonatal rat CM (p=0.035).

### IL-6 Secretion Increases During Ischemia (FIG. 5)

Il-6 levels measured using ELISA in HFCM supernatants were indicative of a trend toward increased IL-6 release during ischemia, and a decline to near control levels during reperfusion, although the differences did not reach statistical significance in the limited sample sizes.

### IL-6 Signaling In HFCM Is Uncoupled During Ischemia/Reperfusion

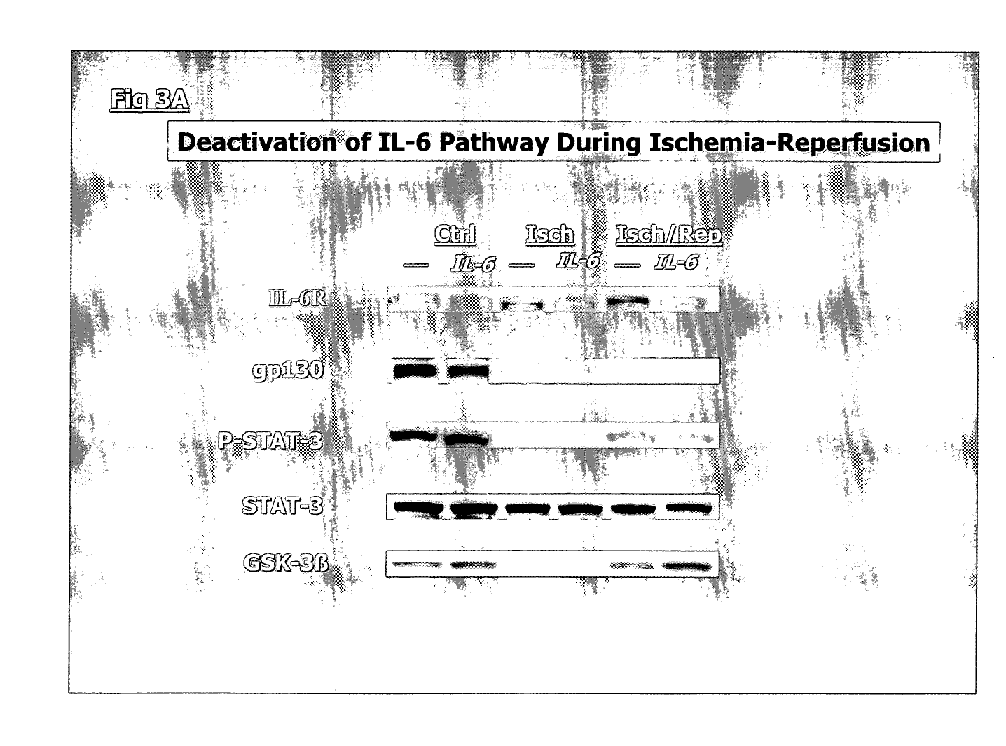

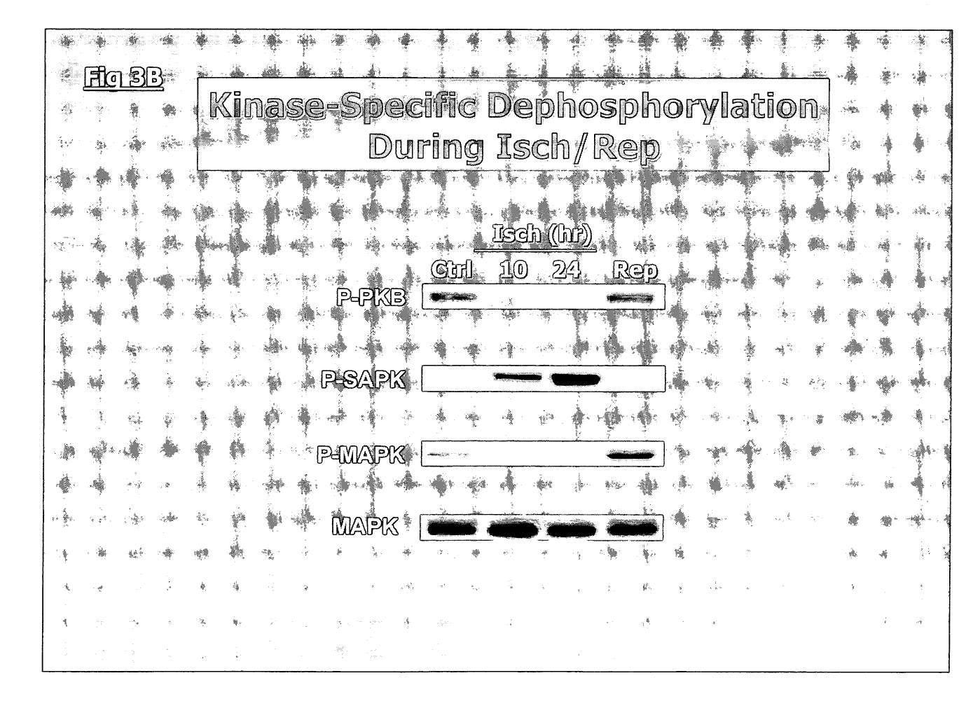

IL-6Rα in HFCM is expressed at low levels under control conditions, increases during 6 hr. ischemia, and to still higher levels following 3 hr. reperfusion (FIG. 3A). STAT-3 is highly phosphorylated under control conditions, becomes almost completely dephosphorylated during ischemia, and is rephosphorylated to intermediate levels following reperfusion. Since STAT-3 is activated by gp130 receptor ligation, the conspicuous decrease in gp130 during I/R is consistent with the correspondent dephosphorylation of STAT-3. Unexpectedly, the addition of IL-6 resulted in a decrease in the levels of IL-6Rα, gp130 and PY705-phosphorylated STAT-3 during ischemia and following reperfusion. This finding may reflect counter-regulatory degradation of the IL-6R following ligation by exogenously-added soluble IL-6. There was a commensurate dephosphorylation of GSK-3β at Ser-9, which represents an activating modification for this classically anti-hypertrophic kinase. The addition of IL-6 increased the extent of GSK-3β phosphorylation under control conditions and following reperfusion. Taken together, this data indicates post-translational inhibition of IL-6 signaling during ischemia-reperfusion, and accords with the corresponding observed decrease in IL-6 message levels (FIG. 5).

### Deactivation of PKB/Akt And MAPK Signaling In HFCM During Ischemia-Reperfusion

The relay system that transmits signals from gp130 to the nucleus involves at least three distinct pathways of protein phosphorylation: the JAK/STAT, PI3-K and the Raf-1/MEK/MAPK pathways. Western analysis indicates dephosphorylation of PKB/Akt at Ser-473 at 10 hr. of ischemia (FIG. 3B), although sequential measurements indicated an easily detectable loss of phosphorylation within 30 minutes of ischemia (data not shown). A decline of similar magnitude in the phosphorylation of the p^{42/44} isoform of MAPK was evident during ischemia, with partial reperfusion-mediated rephosphorylation (FIG. 3B). In concert with the reduction in MAPK message levels by microarray analysis (FIG. 5), this post-translational modification would predict deactivation of MAPK-mediated signaling during ischemia-reperfusion. The finding that the stress-activated serine-threonine kinase/c-jun N-terminal kinase (SAPK/JNK), exhibited an increase in the (T183/Y185) phosphorylation signal during ischemia (FIG. 3B), indicates that the observed modifications in MAPK, PKB/Akt and GSK-3β do not simply reflect non-specific global protein dephosphorylation events. Since activation of SAPK/JNK has been linked to IL-6 gene expression on the basis of gene disruption in mouse embryonic fibroblasts, the lack of activation of this kinase during reperfusion is consistent with the generalized and concomitant repression of IL-6 signaling demonstrated in the human fetal CM.

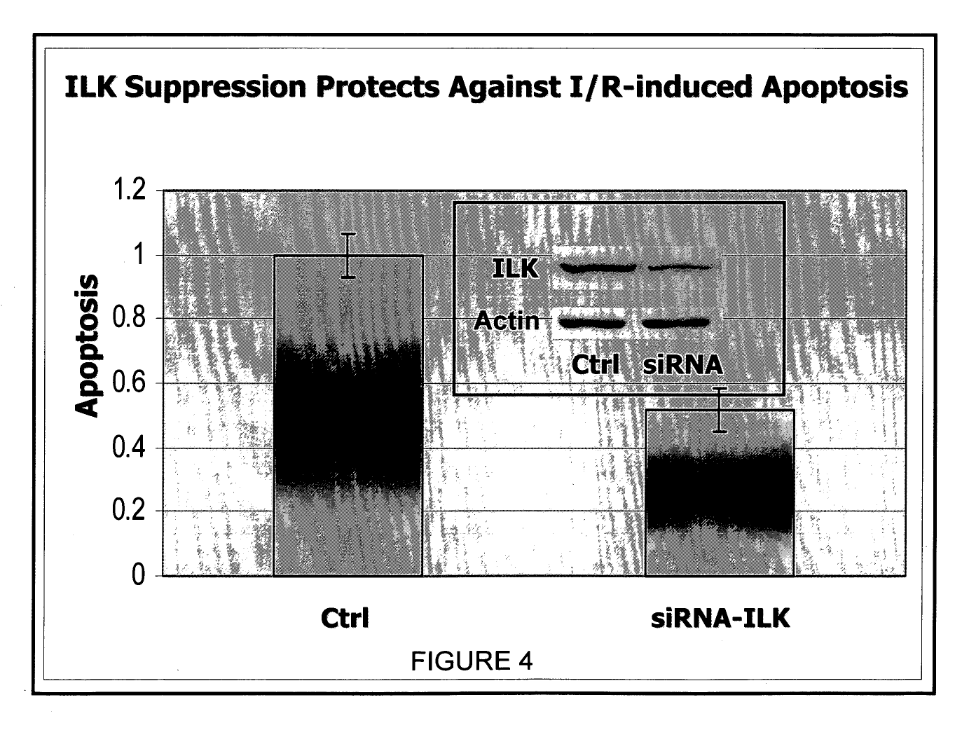

### ILK Knockdown Protects Against Cardiomyocyte Stress-Induced Apoptosis (FIG. 4)

Integrin-linked kinase (ILK) is a novel pro-hypertrophic kinase which causes phosphorylation of PKB/Akt and GSK-3β. We asked whether siRNA-mediated suppression of this pro-proliferative kinase, which should mimic the signaling effects observed in the fetal CM, could influence apoptotic threshold in the neonatal rat CM during I/R. As shown in FIG. 4(insert), lipofectamine-mediated transfection of the ILK-specific siRNAs but not the GAPDH siRNA resulted specifically in substantial (42%; p=0.02) knockdown of ILK expression in neonatal rat cardiocytes as determined by Western blot analysis at 72 hr. post-transduction. Exposure of ILK-silenced NRCM to 6 hr. ischemia and 3 hr reperfusion resulted in an approximate 50% decrease (p=0.031) in the apoptosis rate in comparison to lipofectamine-only controls.

### Discussion

A major finding in the present study is that the human fetal cardiomyocyte exhibits resilience against pro-apoptotic stimuli. This was evident in the relative attenuation of cardiomyocyte apoptosis, in comparison to that in a more mature cellular phenotype, in response to simulated ischemia-reperfusion and to exogenous IL-6 exposure. The transcript profile induced by simulated ischemia-reperfusion in the fetal CM reveals several putative molecular targets which may account for this innately cytoprotective phenotype. We have previously shown that stress exposure elicits a compensatory stress-specific transcriptional response. This was based on the finding, in neonatal patients with hypertrophic congenital heart disease, of a dominant anti-hypertrophic transcriptional profile which appeared to be proportionate to the severity of hypertrophy. By analogy, therefore, we propose and herein provide evidence for the idea that exposure of the human fetal CM to pro-apoptotic stimuli, specifically ischemia-reperfusion injury, can be used to identify compensatory anti-apoptotic molecular responses.

Genome expression profiling in the fetal CM revealed a conspicuous repression, or lack of induction, of IL-6 transcription during ischemia and especially during reperfusion (58% of pre-ischemic levels by microarray; 25% by qPCR). IL-6 is a multifunctional cytokine with pro-proliferative and pro-hypertrophic properties in the heart. Up-regulation of serum and myocardial levels of pro-inflammatory cytokines [tumor necrosis factor (TNF)-α, interleukin-1; and IL-6] have been reported in infants with tetralogy of Fallot, and increased IL-6 message is found in ischemic/reperfused rat heart. IL-6 and its specific receptor (IL6Rα) are also up-regulated in the failing myocardium, and both are down-modulated in the process of favorable cardiac remodeling after left ventricular assist device implantation. However, the specific alterations in the IL-6 signaling pathway induced in the human CM during ischemia-reperfusion are unknown, and it is unresolved in the literature as to whether stress-induced elevation in circulating IL-6 represents a cardiomyocyte-protective or -injurious response.

Two lines of evidence in the present application suggest that repression of IL-6 signaling observed in the human fetal CM represents a protective anti-apoptotic response. First, repression of IL-6 signaling is evident in the fetal CM, which is shown to exhibit an inherently anti-apoptotic phenotype. Secondly, acute exogenous IL-6 exposure designed to mimic clinical disease states caused apoptosis in both human fetal and rat CM which was dose-dependent, evident under control conditions and amplified during ischemia.

The instant results indicate that the IL-6 pathway is inhibited at multiple levels of regulation in HFCM in response to ischemia-reperfusion, including gp130 receptor expression, STAT-3 phosphorylation, and IL-6 transcription. Reduced STAT-3 phosphorylation and IL-6 transcription could result from down-regulation of gp130 expression, since this represents the proximal signaling module of the IL-6 cascade, despite the finding of a concomitant increase in the IL-6Rα subunit during both the ischemic and reperfusion phases. The reason for down-regulation of gp130 is unknown. One may speculate that ischemia-induced disruption of membrane lipid rafts plausibly accounts for this finding, since IL-6 receptor complex localization and STAT-3 signal transduction are raft-dependent. Proteolytic release of the ectodomain of the membrane-bound IL-6R could also explain the increase in IL-6 release observed during ischemia, which may represent a degradation product of the IL-6-ligated IL-6R complex.

The finding that IL-6 potentiates stress-induced CM apoptosis appears to conflict with previous reports demonstrating cardioprotective properties of this cytokine. It may be speculated, however, that the mitogenic, pro-hypertrophic state associated with IL-6 stimulation may be energetically unfavorable under conditions of severe oxygen deprivation, especially since IL-6 signaling has been linked to the generation of reactive oxygen species. The use of unbiased genome profiling provides further support for the idea that the human fetal CM acquires a quiescent phenotype in response to oxygen restriction, including the finding of a significant reduction in the expression of mitogen-activated protein kinase 1 {Locus Link aliases: MAPK1, extra-cellular signal-related kinase-2 (ERK2), p42} (FIG. 5). Activation of the MAP kinase cascade promotes an activating phosphorylation of the nuclear factor for IL-6 (NF-IL-6), also termed C/EBP(, a member of the CCAAT/enhancer binding protein (c/EBP) family of transcription factors. NF-IL-6 binds to a 23-bp DNA element in the IL-6 promoter termed the multiple response element, which is essential for induction of IL-6 transcription after treatment with TNF- (and IL-1.

IL-6 induces cell proliferation by phosphorylation of the 4E-BP1 translational repressor through an ERK-dependent pathway in multiple myeloma cells, a mechanism which may also account for angiotensin II-induced mitogenic responses observed in cardiac fibroblasts and myofibroblasts. It is also noteworthy that platelet-derived growth factor receptor alpha enhances downstream MAPK phosphorylation in a dose-dependent manner in medulloblastoma, in light of our finding of reduced levels of this growth factor transcript during reperfusion (FIG. 5). Thus, de-induction of the MAPK signaling cascade, resulting from both decreased transcript and protein phosphorylation levels observed in the human fetal CM, may serve the energetically beneficial purpose of dampening agonist-induced, pro-inflammatory and pro-proliferative signaling during ischemia and reperfusion. IL-6 signaling may involve PI3-K-dependent as well as STAT-3 and MAPK pathways. Our data also indicates that deactivation of the PI3-K-dependent kinase, PKB/Akt, occurs in the HFCM during ischemia-reperfusion, thereby nullifying potential activation of IL-6 signaling via this collateral pathway.

Integrin-linked kinase (ILK) is a pro-hypertrophic kinase which is regulated in a PI3-K-dependent manner following distinct signal inputs from integrins and growth factor receptor tyrosine kinases. ILK causes phosphorylation of PKB/Akt and GSK-3βp-post-translational modifications which are diametrically opposite to that which was observed in HFCM exposed to ischemic stress. Therefore, we used siRNA-mediated silencing of ILK in order to recapitulate the ‘fetal’ response in the more apoptosis-prone rat CM model. This manipulation revealed an anti-apoptotic effect of ILK knockdown, and provides additional indirect evidence that inhibition of pro-hypertrophic signaling may represent a cardioprotective strategy under conditions of severe oxygen deprivation. This result, however, is antithetical to previous reports which demonstrate that enforced, adenovirally-mediated activation of PI3-K and PKB/Akt exert a protective effect against in vitro and in vivo ischemia-reperfusion injury. The reasons for the discrepant results are unknown but may result from species or model differences, including the use in our study of anoxia rather than hypoxia or ischemia. This suggests, however, that the modulation of classical pro-survival kinases to minimize cardiomyocyte apoptosis may depend on the presence and severity of oxygen deprivation. Further, deactivation of ILK used on our study would presumably affect a unique, non-PKB/Akt-dependent subset of genes, and suggests an unprecedented avenue of cardioprotection.

The induction of the anti-oxidant gene, metallothionein I, observed in the fetal CM transcriptional profile during ischemia-reperfusion, suggests an additional explanation for the apoptosis-resistance evident in this cellular phenotype, since this anti-oxidant has been shown to directly potentiate anti-apoptotic signal transduction as well as mitigate redox-mediated injury. The functional significance of other differentially expressed genes identified in this study (FIG. 5), either singly or in combinatorial permutations, will require further experimentation conducted in both vulnerable and apoptosis-resistant cardiomyocyte phenotypes involving gene-specific gain- and loss-of-function strategies.

Taken together, our data support the general conclusion that the human fetal cardiac myocyte represents a useful biosynthetic platform for the identification of innate cardioprotective responses at the molecular level. The adaptive stress response exemplified by the fetal cardiomyocyte implicates the IL-6 pathway as an important and therapeutically-relevant arbiter of survival.

□ HYPERLINK “http://Names” □□Names□ and identifications of all nucleic acid sequences, inclusive of EST's which are part of the instantly disclosed fetal gene expression profile were deduced via the UNIGENE data bank, and each of the individual UNIGENE Cluster ID reports and all related citations and sequence listings associated therewith are herein incorporated by reference, as if they were a part of the original specification.

Significant genes were searched using The Stanford Online Universal Resource for Clones and ESTs (SOURCE), which compiles information from several publicly accessible databases, including UniGene, dbEST, Swiss-Prot, GeneMap99, RHdb, GeneCards and LocusLink.

All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as. well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

### FILES REFERENCED IN SPECIFICATION

1 Shi Y, Baker J E, Zhang C, Tweddell J S, Su J, Pritchard K A Jr. Chronic hypoxia increases endothelial nitric oxide synthase generation of nitric oxide by increasing heat shock protein 90 association and serine phosphorylation. Circ Res 2002; 91 (4):300-306.
2 Konorev E, Tarpey M, Joseph J, Baker J, Kalyanaraman B. S-nitrosoglutathione improves functional recovery in the isolated rat heart after cardioplegic ischemic arrest-evidence for a cardioprotective effect of nitric oxide. J Pharmacol Exp Ther 1995; 274:200-206.
3 Madan A, Varma S, Cohen H J. Developmental stage-specific expression of the alpha and beta subunits of the HIF-1 protein in the mouse and human fetus. Molecular Genetics and Metabolism 2002; 75 (3):244-249.
4 Hauck L, Hansmann G, Dietz R, von Harsdorf R. Inhibition of hypoxia-induced apoptosis by modulation of retinoblastoma protein-dependent signaling in cardiomyocytes. Circ Res 2002; 91 (9):782-789.
5 Hammel J M, Caldarone C A, Van Natta, et al. Myocardial apoptosis after cardioplegic arrest in the neonatal lamb. J Thorac Cardiovasc Surg 2003; 125:1268-75.
6 Friehs 1, Cao-Dank H, Stamm C, Cowan D B, McGowan F X, del Nido P J. Postnatal increase in insulin-sensitive glucose transporter expression is associated with improved recovery of postischemic myocardial function. J Thorac Cardiovasc Surg 2003; 126 (1):263-271.
7 Karimi M, Wang L X, Hammel J M, Mascio C E, Abdulhamid M, Barner E W, Scholz T D, Segar J L, Li W G, Niles S D, Caldarone C A. Neonatal vulnerability to ischemia and reperfusion: Cardioplegic arrest causes greater myocardial apoptosis in neonatal lambs than in mature lambs. J Thorac Cardiovasc Surg 2004; 127 (2):490-497.
8 Konstantinov I E, Coles J G, Boscarino C, Takahashi M, Goncalves J, Ritter J, Van Arsdell G S. Gene expression profiles in children undergoing cardiac surgery for right heart obstructive lesions. J Thorac Cardiovasc Surg 2004; 127 (3):746-754.
9 Ancey C, Menet E, Corbi P, Fredj S, Garcia M, Rucker-Martin C, Bescond J, Morel F, Wijdenes J, Lecron J C, Potreau D. Human cardiomyocyte hypertrophy induced in vitro by gp130 stimulation. Cardiovasc Res 2003; 59 (1):78-85.
10 Zolk O, Ng L L, O'Brien R J, Weyand M, Eschenhagen T. Augmented expression of cardiotrophin-1 in failing human hearts is accompanied by diminished glycoprotein 130 receptor protein abundance. Circulation 2002; 106:1442-1446.
11 Corbi P, Rahmati M, Delwail A, Potreau D, Menu P, Wijdenes J, Lecron J C. Circulating soluble gp130, soluble IL-6R, and IL-6 in patients undergoing cardiac surgery, with or without extracorporeal circulation. Eur J Cardiothorac Surg 2000; 18:98-103.
12 Grotzinger J, Kemebeck T, Kallen K J, Rose-John S. IL-6 type cytokine receptor complexes: hexamer, tetramer or both? Biol Chem 1999:380:803-813.
13 Heinrich P C, Behrmann I, Muller-Newen G, Schaper F, Graeve Interleukin-6-type cytokine signalling through the gp130/Jak/STAT pathway. Biochem J 1998; 334:297-314.
14 Kubasiak L A, Hernandez O M, Bishopric N H, Webster K A. Hypoxia and acidosis activate cardiac myocyte death through the Bcl-2 family protein BNIP3. Proc Natl Acad Sci USA 2000; 99:12825-12830.
15 Vitadello M, Penzo D, Petronilli V, Michieli G, Gomirato S, Menabo R, Di Lisa F, Gorza L. Overexpression of the stress protein Grp94 reduces cardiomyocyte necrosis due to calcium overload and simulated ischemia. FASEB J 2003; 17:923-925.
16 Craig R, Wagner M, McCardle T, Craig A G, Glembotski C C. The cytoprotective effects of the glycoprotein 130 receptor-coupled cytokine, cardiotrophin-1, require activation of NF-kappa B. J Biol Chem 2001; 276 (40):37621-37629.
17 Tusher V G, Tibshiran R, Chu G. Significance analysis of microarrays applied to the ionizing radiation response. PNAS 2001; 98 (9):5116-5121.
18 Holloway A J, vanLaar R K, Tothill R W, Bowtell D D L. Options available-from start to finish for obtaining data from DNA microarrays II. Nature Genetics 2002; 32:481-489.
19 Ito T, Ikeda U, Shimpo M, Ohki R, Takahashi M, Yamamoto K, Shimada K. HMG-CoA reductase inhibitors reduce interleukin-6 synthesis in human vascular smooth muscle cells. Cardiovasc Drugs Ther 2002; 16 (2):121-126.
20 Altschul S F, Gish W, Miller W, Myers E W, Lipman D J. Basic local alignment search tool. J Mol Biol 1990; 215 (3):403-410.
21 Kumar A S, Naruszewicz I, Wang P, Leung-Hagesteijn C, Hannigan G E. ILKAP regulates ILK signaling and inhibits anchorage-independent growth. Oncogene 2004; March: 1-4.
22 Antos C L, McKinsey T A, Frey N, Kutschke W, McAnally J, Shelton J M, Richardson J A, Hill J A, Olson E N. Activated glycogen synthase-3 beta suppresses cardiac hypertrophy in vivo. Proc Natl Acad Sci USA 2002; 99 (2):907-912.
23 Frey N, McKinsey T A, Olson E N. Decoding calcium signals involved in cardiac growth and function. Nat Med 2000; 6:1221-1227.
24 Kunisada K, Hirota H, Fujio Y, Matsui H, Tani Y, Yamauchi-Takihara K, Kishimoto T. Activation of JAK-STAT and MAP kinases by leukemia inhibitory factor through gp130 in cardiac myocytes. Circulation 1996; 94:2626-2632.
25 Oh H, Fujio Y, Kunisada K, Hirota H, Matsui H, Kishimoto T, Yamauchi-Takihara K. Activation of phosphatidylinositol 3-kinase through glycoprotein 130 induces protein kinase B and p70 S6 kinase phosphorylation in cardiac myocytes. J Biol Chem 1998; 273:9703-9710.
26 Nishitai G, Shimizu N, Negishi T, Kishimoto H, Nakagawa K, Kitagawa D, Watanabe T, Momose H, Ohata S, Tanemura S, Asaka S, Kubota J, Saito R, Yoshida H, Mak T W, Wada T, Penninger J M, Azuma N, Nishina H, Katada T. Stress induces mitochondria-mediated apoptosis independent of SAPK/JNK activation in embryonic stem cells. J Biol Chem 2004; 279 (3):16211626.
27 Hannigan G E, Leung-Hagesteijn C, Fitz-Gibbon L, Coppolino M G, Radeva G, Filmus J, Bell J C, Dedhar S. Regulation of cell adhesion and anchorage-dependent growth by a new beta 1-integrin-linked protein kinase. Nature 1996; 379:91-96.
28 Troussard A A, Tan C, Yoganathan N, Dedhar S. Cell-extracellular matrix interactions stimulate the AP-1 transcription factor in an integrin-linked kinase- and glycogen synthase kinase 3-dependent manner. Mol Cell Biol 1999; 19:7420-7427.
29 Troussard A A, Mawji N M, Ong C, Mui A, St-Arnaud R, Dedhar S. Conditional knock-out of integrin-linked kinase demonstrates an essential role in protein kinase B/Akt activation. J Biol Chem 2003; 278 (25):22374-22378.
30 Stockand J D, Meszaros J G. Aldosterone stimulates proliferation of cardiac fibroblasts by activating Ki-RasA and MAPKI/2 signaling. Am J Physiol Heart Circ Physiol 2003; 284:H176H184.
31 Hunter J J, Chien K R. Signaling pathway for cardiac hypertrophy and failure. New Engl J Med 1999; 341:1276-1283.
32 Hovels-Gurich H H, Schumacher K, Vazquez-Jimenez J F, Qing M, Huffineier U, Buding B, Messmer B J, von Bernuth G, Seghaye M C. Cytokine balance in infants undergoing cardiac operation. Ann Thorac Surg 2002; 73 (2):601-608.
33 Qing M, Schumacher K, Heise R, Woltje M, Vazquez-Jimenez J F, Richter T, Arranda-Carrero M, Hess J, Bernuth G, Seghaye M-C. Intramyocardial synthesis of pro- and anti-inflammatory cytokines in infants with congenital cardiac defects. J Amer Coll Cardiol 2003; 41 (12):22662274.
34 Onody A, Zvara A, Hackler L Jr, Vigh L, Ferdinandy P, Puskas L G. Effect of classic preconditioning on the gene expression pattern of rat hearts: a DNA microarray study. FEBS Letters 2003; 536:35-40.
35 Plenz G, Baba H A, Erren M, Scheld H H, Deng M C. Reversal of myocardial interleukin-6 mRNA expression following long-term left ventricular assist device support for myocarditis-associated low output syndrome. J Heart Lung Transplant 1999; 18:923-924.
36 Kim J, Adam R M, Solomon K R, Freeman M R. Involvement of cholesterol-rich lipid rafts in interleukin-6-induced neuroendocrine differentiation of LNCaP prostate cancer cells. Endocrinology 2004; 145 (2):613-619.
37 Matthews V, Schuster B, Schutze S, Bussmeyer 1, Ludwig A, Hundhausen C, Sadowski T, Saftig P, Hartmann D, Kallen K J, Rose-John S. Cellular cholesterol depletion triggers shedding of the human interleukin-6 receptor by ADAM10 and ADAM17 (TACE). J Biol Chem 2003; 278 (40):38829-38839.
38 Pennica D, King K L, Shaw K J, Luis E, Rullamas J, Luoh S M, Darbonne W C, Knutzon D S, Yen R, Chien K R, et al. Expression cloning of cardiotrophin 1, a cytokine that induces cardiac myocyte hypertrophy. Proc Natl Acad Sci USA 1995; 92 (4):1142-1146.
39 Hirota H, Chen J, Betz U A, Rajewsky K, Gu Y, Ross J Jr, Muller W, Chien K R. Loss of a gp130 cardiac muscle cell survival pathway is a critical event in the onset of heart failure during biomechanical stress. Cell 1999; 97 (2):189-198.
40 Dong Y, Benveniste E N. Immune function of astrocytes. Glia 2001; 36:180-190.
41 Hattori T, Ohoka N, Hayashi H, Onozaki K. C/EBP homologous protein (CHOP) up-regulates IL-6 transcription by trapping negative regulating NF-IL6 isoform. FEBS Letter 2003; 541:33-39.
42 Isshiki H, Akira S, Tanabe O, Nakajima T, Shimamoto T, Hirano T, Kishimoto T. Constitutive and interleukin-1 (IL-1)-inducible factors interact with the IL-1-responsive element in the IL-6 gene. Mol Cell Biol 1990; 10:2757-2764.
43 Shi Y, Hsu J-H, Hu L, Gera J, Lichtenstein A. Signal pathways involved in activation of p70S6K and phosphorylation of 4E-BP 1 following exposure of multiple myeloma tumor cells to interleukin-6. J Biol Chem 2002; 277:15712-15720.
44 MacDonald T J, Brown K M, LaFleur B, Peterson K, Lawlor C, Chen Y, Packer R J, Cogen P, Stephan D A. Expression profiling of medulloblastoma: PDGFRA and the RAS/MAPK pathway as therapeutic targets for metastatic disease. Nat Genet 2001; 29:143-52.
45 Attwell S, Roskelley C, Dedhar S. The integrin-linked kinase (ILK) suppresses anoikis. Oncogene 2000; 19:3811-3815.
46 White D E, Cardiff R D, Dedhar S, Muller W J. Mammary epithelial-specific expression of the integrin-linked kinase (ILK) results in the induction of mammary gland hyperplasias and tumors in transgenic mice. Oncogene 2001; 20:7064-7072.
47 Matsui T, Tao J, del Monte F, Lee K H, Li L, Picard M, Force T L, Franke T F, Hajjar R J, Rosenzweig A. Akt activation preserves cardiac function and prevents injury after transient cardiac ischemia in vivo. Circulation 2001; 104:330-335.
48 Matsui T, Li L, del Monte F, Fukui Y, Franke T F, Hajjar R J, Rosenzweig A. Adenoviral gene transfer of activated phosphatidylinositol 3′-kinase and Akt inhibits apoptosis of hypoxic cardiomyocytes in vitro. Circulation 1999; 100:2373-2379.

What is claimed is:

1. A fetal gene expression network expressed in human fetal cardiac myocytes as a result of ischemia/reperfusion consisting essentially of:
Hs. 74615, Hs. 433989, Hs. 179573, Hs. 119129, Hs. 87409, Hs. 458104, Hs. 274464, Hs. 93913, Hs. 75636, Hs. 344080, Hs. 185973, Hs. 348389, Hs. 90998, Hs. 168159, Hs. 246381, Hs. 91299, Hs. 3094, Hs.433205, Hs.457574, Hs.8364, Hs.405944, Hs.117848, Hs.76847, Hs.102950, Hs.194019, Hs.82646, Hs.108972, Hs.154078, Hs.372513, Hs.227656, Hs.28805, and Hs.85155.

2. The fetal gene expression network of claim 1, wherein said network is associated with an apoptosis resistance phenotype.

3. The fetal gene expression network of claim 1, wherein said network is associated with repression of IL-6 signaling.
